(19) United States
(12) Patent Application Publication
(10) Pub. No.: US 2016/0068797 A1
(43) Pub. Date: Mar. 10, 2016

(54) METHOD AND APPARATUS FOR FILLING A PLURALITY OF MEDIA PLATES IN A SELF-SUPPORTING STACK

(71) Applicants: SINGER INSTRUMENT COMPANY LTD, Watchet Somerset (GB); Kreo Technologies Inc., Oakville (CA)

(72) Inventors: Pawel KUZAN, Oakville (CA); Harry SINGER, Watchet (GB)

(21) Appl. No.: 14/787,018

(22) PCT Filed: Apr. 25, 2014
(86) PCT No.: PCT/GB2014/051287

§ 371 Date: Oct. 26, 2015

(30) Foreign Application Priority Data
Apr. 26, 2013 (GB) 1307626.0

(51) Int. Cl. C12M 1/34; B65B 3/26; C12M 1/00; B65B 3/04

(52) U.S. Cl. CPC C12M 41/44; B65B 3/04; B65B 3/26; C12M 29/00

(57) ABSTRACT

A method and apparatus for filling one or more of a plurality of media plates each with a given quantity of media fluid. The media plates may be arranged in a self- supporting stack. The method comprises moving a carriage along a height of the stack of media plates to a position of a media plate to be filled and causing gripping means of said carriage to grip a lid of said media plate or, if said media plate has no lid, to grip a media plate directly above said media plate to be filled. The carriage is caused to rise to lift said lid, if present, and lift any media plates arranged above said media plate to be filled to provide access to an interior of said media plate. The interior, i.e. basin, of said media plate is filled with a given quantity of media fluid such as agar which may subsequently solidify to provide a growth medium for biological agents such as bacteria, yeast or the like. The carriage is caused to descend to replace said lid, if present, and return any lifted media plates to their former positions arranged above said media plate to be filled. The method does not require a media plate to be removed from the stack in order to fill it.

The invention relates generally to a method and apparatus for filling a plurality of media plates in a self-supporting stack and more particularly, but not exclusively, to filling a plurality of media plates in a self-supporting stack by working up the plurality of media plates in turn starting from the lowermost plate in the stack.

### BACKGROUND

Most conventional plate pourer apparatuses work on the same general principle which is described in U.S. Pat. No. 4,170,861. Stacks of Petri dishes or plates are loaded into a magazine, for example a rotating turret, which sits on top of a plate-handling apparatus base. A mechanism at a dish-drop station lowers one dish from the bottom of a stack of empty dishes into a filling chamber. As the dish is lowered, the lid or cover is separated from the dish. A turntable transfers the dish and lid from the dish-drop station to the filling station, where the media fluid, e.g. liquid agar, is dispensed into the dish through a fixed horizontal nozzle. Once the dish is filled, the dish and the lid are transferred to an elevating station, where the dish is raised to close it with the lid and then pushed into the bottom of a stack of already filled dishes. The entire stack of filled dishes is moved up when another dish is added.

The conventional plate pourer apparatuses of this type are designed to handle only round Petri dishes. There do not appear to be any commercially available plate pourer apparatuses which can handle non-Petri dish media plates, e.g. rectangular or square plates which are increasingly being used, especially in automated sampling apparatuses. Furthermore, the conventional plate pourer apparatuses appear to be able to handle only one specific size, i.e. diameter, of Petri dishes, typically 100 mm or 60 mm. Some apparatuses can be re-tooled to handle another Petri dish size, but switching to a different plate size normally requires stopping the apparatus and replacing the plate magazine and several other components of the apparatus.

A consequence of the plate handling method used in most conventional apparatuses is that the apparatuses can accept plates only within a very narrow range of diameters very close to their “nominal” size, which severely limits the choice of consumables. Moreover, spills from plates within the apparatuses are often very difficult to clean as the plate stacks tends to sit directly on top of the plate pourer's main body, which contains most moving parts, electronics, etc. In addition to being difficult to clean and sterilize, the media fluid filling chamber is often not completely enclosed, which introduces the risk of contaminating the media with airborne microorganisms.

U.S. Pat. No. 4,468,914 discloses an apparatus for filling Petri dishes or plates whereby plates to be filled are moved continuously from a stack towards a dispensing nozzle. The covers or lids of the plates engage a ramp which tilts said lids away from the plates to provide access to an interior of the plates for filling purposes. After the plates are filled, their lids are dropped back into place.

U.S. Pat. No. 3,719,023 describes a similar plate filling apparatus to U.S. Pat. No. 4,468,914.

U.S. Pat. No. 3,704,568 describes an apparatus for filling Petri dishes in which a dish and a lid are fed forward in rectilinear paths in different levels from a stack of closed dishes in a hopper at one end of a sterile chamber, the dish being caused to drop down on the bottom of the chamber between slide rails that catch the edge of the lid. In one position of the travelling path the dish is filled with substrate, and in a subsequent position the lid is caused to drop down on the dish.

It can be seen that all of the above described apparatuses require plates to be moved from a stack for filling and/or returned to a stack after filling.

### GENERAL

Preferably the invention mitigates or obviates to some degree one or more problems associated with known plate pourer apparatuses.

This may be met by the combination of features of the main claims; the sub-claims disclose further advantageous embodiments of the invention.

Preferably the invention provides a method and apparatus for pouring media into any type and/or size of media plates without requiring the media plate to be removed from a stack for filling or returned to a stack after filling.

Preferably the invention provides a method and apparatus for pouring media into any type and/or size of standard media plates without stopping the apparatus and without having to make any hardware or software modifications or adjustments to the apparatus so that the apparatus can handle plates that differ from their nominal size, as is commonly encountered with commercially available consumables.

Preferably the invention assures that airborne contaminants are prevented from entering the open plates during pouring.

Preferably the invention produces excellent surface quality of the solidified media with uniform thickness and wall-to wall flatness and therefore to eliminate disturbing already poured plates while remaining plates are processed.

One skilled in the art will derive from the following description other objects of the invention. Therefore, the foregoing statements of object are not exhaustive and serve merely to illustrate some of the many objects of the present invention.

### SUMMARY

In a first main aspect, the invention may provide a method of filling one or more of a plurality of media plates each with a given quantity of media fluid. The media plates may be arranged in a self-supporting stack. The method comprises moving a carriage along a height of the stack of media plates to a position of a media plate to be filled and causing gripping means of said carriage to grip a lid of said media plate or, if said media plate has no lid, to grip a media plate directly above said media plate to be filled. The carriage is caused to rise to lift said lid, if present, and lift any media plates arranged above said media plate to be filled to provide access to an interior of said media plate. The interior, i.e. basin, of said media plate is filled with a given quantity of media fluid such as agar which may subsequently solidify to provide a growth medium for biological agents such as bacteria, yeast or the like. The carriage is caused to descend to replace said lid, if present, and return any lifted media plates to their former positions arranged above said media plate to be filled.

Consequently, the method does not require a media plate to be removed from the stack in order to gain access to its interior, i.e. in order to fill it. Furthermore, the method does not require a media plate to be returned to the stack after filling. The stack can be considered as being stationary in that, during the automatic filling process, no plates are removed, returned or added to the stack.

Preferably, the method includes performing the steps of the first main aspect for each of a plurality of media plates in the stack and preferably doing so by starting with the lowermost plate and moving to each plate in turn up the height of the stack. Consequently, the only movement of plates in a stack during an automatic filling process is the raising up a short distance of not yet filled plates above already filled plates and a plate being filled and the return of the raised plates to their former positions.

In a preferred embodiment, the carriage is parked at a position above the stack of plates, especially when a program of filling plates is completed. However, in another embodiment the carriage is parked below a support for the stack of media plates. In either embodiment, parking the carriage above or below the stack of media plates makes loading a stack of clean plates or removing a stack of filled plates more efficient than would be the case otherwise.

The apparatus of the invention may comprise a support for a single stack of media plates, but preferably it comprises additional stack support units and the carriage is provided with sufficient gripping means such that the method may include performing the steps of the first main aspect of the invention simultaneously on two or more stacks of media plates. Where the apparatus supports more than one stack of plates, the carriage may be provided with only one media fluid dispensing means such as a nozzle, whereby the dispensing means is movable relative to the carriage and stacked plates so as to be able to fill corresponding plates in different stacks one after the other prior to the step of lowering the carriage to replace said lid, if present, and return any lifted media plates to their former positions.

Preferably, the carriage is provided with means for determining a size and/or a type of a media plate in the stack. This means may comprise contact means which engage the lid of a media plate to be filled, the plate itself or another plate adjacent thereto in order to establish data describing the plate to be filled. Additionally or alternatively, it may comprise non-contact sensor means which establishes data describing the plate to be filled through non-contact means such as optical sensors and/or proximity sensors. The data from the contact means or non-contact sensor means may enable a process controller of the apparatus to determine a size and/or type of the media plate to be filled.

The method may further comprise determining an operating parameter for filling said media plate based on a determined size and/or type of said plate. The operating parameter may comprise one or more of: volume of media to be filled in the plate; pumping speed for media filling; and location of delivery of media to the interior of the plate.

In a second main aspect, the invention provides an apparatus for filling one or more of a plurality of media plates each with a given quantity of media fluid. The apparatus comprises: a media plate stack support on which a plurality of media plates can be arranged in a self-supporting stack; a carriage movable along a height of the stack of media plates to a position of a media plate to be filled, the carriage carrying gripping means for gripping a lid of said media plate or, if said media plate has no lid, to grip a media plate directly above said media plate to be filled. The carriage is arranged to rise to lift said lid, if present, and lift any media plates arranged above said media plate to be filled to provide access to an interior of said media plate. A media fluid dispensing means is provided for filing the interior of said media plate with a given quantity of media fluid. Once said dispensing means has dispensed a given quantity of media fluid to the interior of the media plate, said carriage is arranged to descend to replace said lid, if present, and return any lifted media plates to their former positions arranged above said media plate to be filled.

In a third main aspect, the invention provides a method of filling a media plate with a given quantity of media fluid using a media plate filling apparatus having a process controller for controlling operations of the apparatus, the method comprising the steps of: automatically determining a size and/or a type of a media plate to be filed; determining an operating parameter for filling said media plate based on the determined size and/or type of said plate; and filling said media plate with a given quantity of media fluid in accordance with said determined operating parameter.

Preferably, the steps of automatically determining a size and/or a type of a media plate and determining an operating parameter comprise: arranging gripping means of said apparatus to engage a lid of a media plate to be filled or to engage the media plate itself; receiving at the process controller an input signal from the gripping means; and determining from said input signal a size and/or a type of a media plate to be filled.

In a fourth main aspect, the invention provides an apparatus for filling a media plate with a given quantity of media fluid having a process controller for controlling operations of the apparatus, the apparatus comprising: means for automatically determining a size and/or a type of a media plate to be filed; means for determining an operating parameter for filling said media plate based on the determined size and/or type of said plate; and dispenser means for filling said media plate with a given quantity of media fluid in accordance with said determined operating parameter.

In a fifth main aspect, the invention provides a carriage for a media plate filling apparatus, said carriage comprising: a first gripping member; and a second gripping member; the first and second gripping members defining therebetween a gripping space and being arranged to be movable relative to one another so as to close said space to grip a media plate or a lid of a media plate located in said space, the second gripping member having at least one first gripping finger member and at least two second gripping finger members, said second gripping finger members being arranged on either side of the at least one first gripping finger member and ends of said second gripping finger members being positioned further into the gripping space than an end of the at least one first gripping finger member.

In a sixth main aspect, the invention provides a method of filling a media plate, the method comprising: providing a media plate filling apparatus having the carriage of the fifth main aspect of the invention; and controlling said media plate filling apparatus to perform the steps of the method of the first main aspect of the invention and/or the method of the third main aspect of the invention.

In a seventh main aspect, the invention provides a computer readable medium carrying machine readable instructions executable by a processor of a computer, said machine readable instructions for implementing in a media plate filling apparatus the steps of the method of the first main aspect of the invention and/or the method of the third main aspect of the invention.

The summary of the invention does not necessarily disclose all the features essential for defining the invention; the invention may reside in a sub-combination of the disclosed features.

### BRIEF DESCRIPTION OF THE DRAWINGS

The foregoing and further features of the present invention will be apparent from the following description of preferred embodiments which are provided by way of example only in connection with the accompanying figures, of which:

FIG. 1 is a perspective view of a plate pourer apparatus according to the invention;

FIG. 2 is a top perspective view of a carriage for the apparatus of FIG. 1;

FIG. 3 is a bottom perspective view of a carriage for the apparatus of FIG. 1;

FIG. 4 is a an above perspective view of a front gripping member of the apparatus of FIG. 1;

FIG. 5 is a an above perspective view of a rear gripping member of the apparatus of FIG. 1;

FIG. 6 is a perspective view of a base and elevator means for the apparatus of FIG. 1;

FIG. 7 is a partial view of the carriage showing the gripping members engaging a 60 mm diameter Petri dish lid;

FIG. 8 is a partial view of the carriage showing the gripping members engaging a 90 mm diameter Petri dish lid;

FIG. 9 is a partial view of the carriage showing the gripping members engaging a 150 mm diameter Petri dish lid;

FIG. 10 is a partial view of the carriage showing the gripping members engaging a rectangular lid of a media plate; and

FIG. 11 is a partial view of the main cabinet of the apparatus showing a double stack of rectangular media plates being processed for filling.

### DETAILED DESCRIPTION

The following description is of embodiments by way of example only and without limitation to the combination of features necessary for carrying the invention into effect.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The definition of a self-supporting stack as used herein does not preclude the use of one or more vertical guides such as a vertically extending guide plate or guide rod(s) against which the stack of plates may rest when loaded into the apparatus, the vertical guide also possibly acting as a means to enable a user to accurately position the stack of plates within the apparatus. Generally speaking, a self-supporting stack refers to a stack of media plates placed one on top of another where each upper plate rests upon the lid of a plate directly below it or, if the lower plate does not have a lid, the upper plate rests on a rim of the lower plate.

Reference herein to a dish is a reference to a plate. Reference herein to a cover is a reference to a lid.

The interior of a media plate or dish is considered as comprising the basin formed by the base and side wall(s) of the plate which enable a given quantity of growth media or the like to be received and contained within the plate.

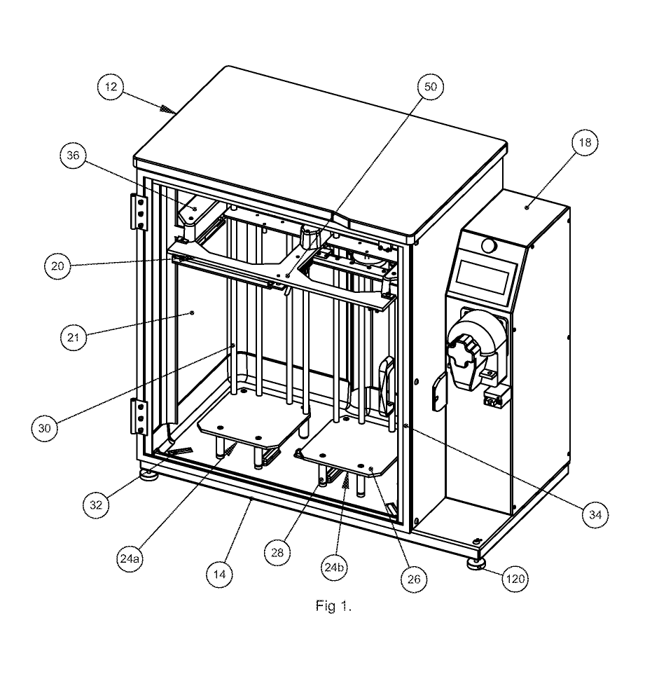

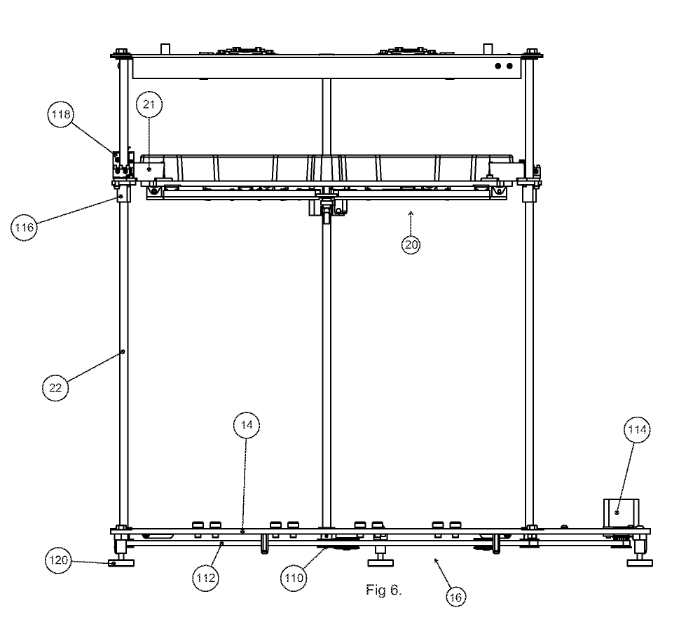

Referring to FIG. 1, a plate pourer apparatus 10 according to the invention comprises a main cabinet 12 having a base 14 with an elevator mechanism 16 (FIG. 6) provided on the underneath of the base 14. A control cabinet 18 is provided on the base 14 besides the main cabinet 12. The control cabinet 18 houses various components including a process controller (not shown) which runs custom software to control the automatic or custom operation of the apparatus 10. The main cabinet 12 contains a carriage 20 supported by carriage mounting brackets 21 (FIG. 6) on three vertical lead screws 22 (FIG. 6), which are hidden behind interior panels 21 of the main cabinet 12. A lead screw (or leadscrew), which is sometimes referred to as a power screw or a translation screw, is a screw designed to translate turning motion into linear motion. The elevator mechanism 16 causes synchronous rotation of the lead screws 22 which translate said rotating motion to linear movement to lower or raise the carriage 20 within the main cabinet 12. It will be understood that other types of elevation mechanism for the carriage 20 are within the scope of the invention.

The cabinet 12 also contains two removable stack support units 24a,b. Each stack support unit 24a, b comprises a support base plate 26 which is adapted to support a stack of media plates in a self-supporting manner. Each support base plate 26 is spaced above the top surface of the base 14 by a short distance, being mounted on legs 28. Each stack support unit 24a,b has one or more vertically extending guide rods 30 adapted to enable a user to position a stack of media plates accurately in the apparatus on one of the support base plates 26. The guide rods 30 extend up through the carriage 20 such that the carriage surrounds the guide rods 30 and a stack of media plates placed adjacent or against the guide rods 30 when the carriage 20 moves along the height of the stack of plates. It will be appreciated that the apparatus 10 may use one or more guide plates rather than guide rods 30, but guide rods 30 are preferred as they give more unhindered access than guide plates to the stack of media plates positioned in the stack support units 24,b. In some embodiments, there are no guide plates or guide rods.

At the top of the cabinets 12, 18 there may be some LED indicator lights and a germicidal UV light providing indications of certain operations of the apparatus 10 to a user.

In the bottom of the cabinet 12 there may be two level indicators 32 visible through transparent sections of a bottom liner of the cabinet 12. A door 34 of the cabinet 12 may have a magnet that activates a safety switch on the cabinet frame as a form of door interlock.

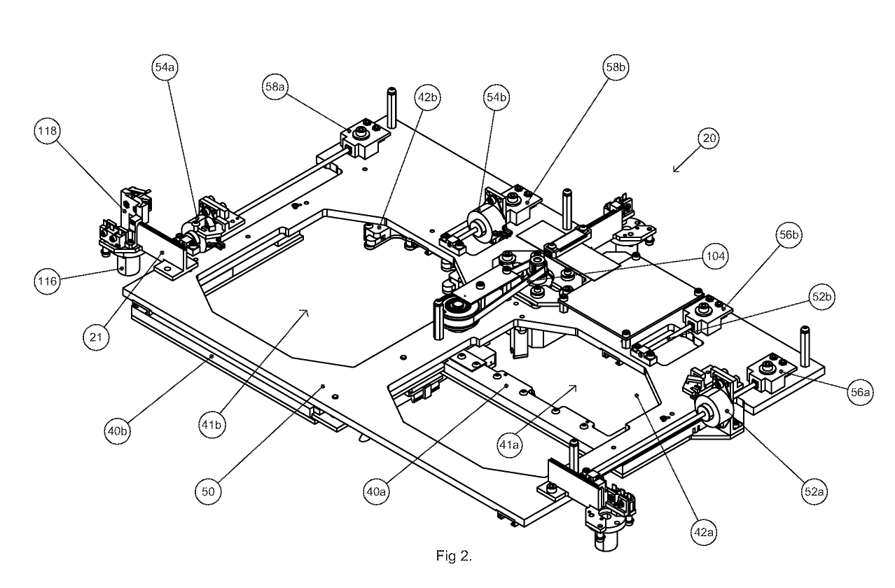

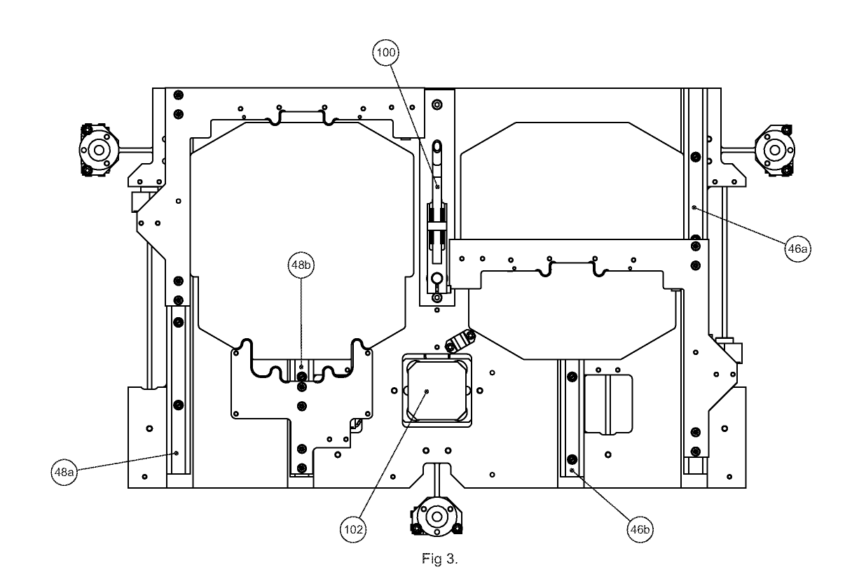

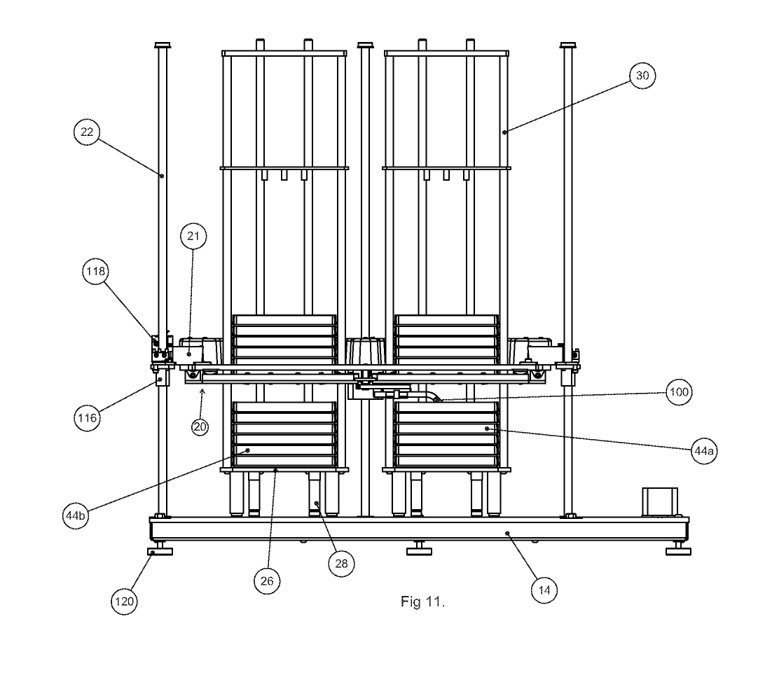

Some working components of the carriage 20 may be enclosed by a cover 36. Referring to FIGS. 2 and 3, it can be seen that the carriage 20 has four plate grippers comprising a first pair of front and rear gripper members 40a,b and a second pair of front and rear gripper members 42a,b that engage right and left (as viewed in the drawings) stacks of plates 44a,b (FIG. 11) respectively. The first pair of gripper members 40a,b define therebetween a first gripping space 41a. The second pair of gripper members 42a,b define therebetween a second gripping space 41b. The gripper members 40a,b, 42a,b are supported on respective rails 46a,b, 48a,b mounted underneath a main plate member 50 of the carriage 20 so as to be movable towards each other. Each of the four gripper members 40a,b, 42a,b is independently actuated by an actuator preferably comprising a dedicated linear stepper motor 52a,b, 54a,b. FIGS. 2 and 3 show the right pair of gripper members 40a,b moved to a fully closed position, and the left pair of gripper members 42a,b in a fully open position. Each gripper actuator is equipped with a force switch 56a,b, 58a,b located towards the back of the main plate 50 that detects overload on the gripper members in either the gripper-closing or gripper-opening directions.

The front gripper members 40a, 42a have a much larger distance of movement from their open to closed positions than the distance of movement of the rear gripper members 40b, 42b. This is because the rear gripper members 40b, 42b are generally located behind the guide rods 30 and advance into the gripping spaces 41a,b by a small distance such that their protrusions 82a,b, 84a,b and finger member protrusions 86a,b, 88a,b extend between the guide rods 30 into the gripping spaces 41a,b in front of the guide rods 30.

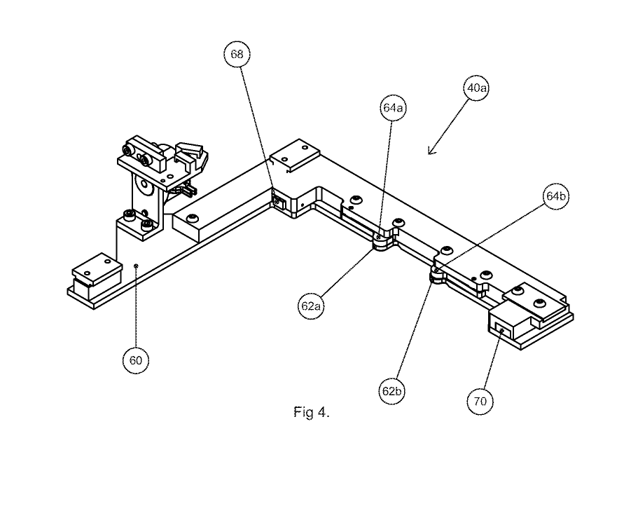

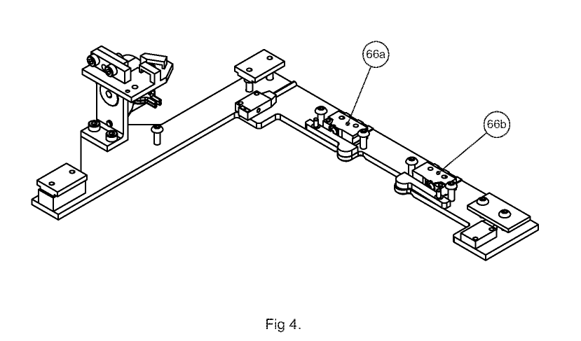

The right-front gripper member 40a as seen in FIG. 4 comprises a generally L-shaped support bracket 60 comprising a thin metal plate with two semi-circular finger-like protrusions 62a,b which in use face the plate stack 44a and can be moved into engagement with a media plate lid or a media plate itself. Directly above these protrusions 62a,b are two movable finger members 64a,b of a matching shape to the protrusions 62a,b. When the front gripper member 40a engages a media plate's lid, the protrusions 62a,b on the gripper support bracket 60 enter the space directly underneath the lid's bottom edge. As the movable finger members 64a,b are pushed by the lid they activate respective finger switches 66a,b which confirm that the gripper member 40a is in position to support and lift the media plate's lid. In FIG. 4, the finger member 62a on the left hand side is shown released and the finger member 62b on the right hand side is shown activated, i.e. pressed by a media plate lid. As an alternative to engaging a media plate lid, the protrusions 62a,b and the finger members 64a,b could be arranged to engage the bottom and sides of a media plate in order to support and lift it. This arrangement is useful where a stack of plates is arranged without plate lids such that a bottom of an upper plate rests on a rim of a lower plate with perhaps only the uppermost plate in the stack having a lid.

In addition to the finger switches 66a,b that provide tactile feedback during plate handling, the right-front gripper member 40a has a photoelectric thru-beam sensor system comprising a receiver 68 and a transmitter 70, which is used to locate the bottom edge of the plate's lid or the bottom edge of the plate before the right pair of gripper members 40a,b are moved towards each other to engage the lid or plate. The transmitter 70 and receiver 68 of this sensor system are mounted on opposite sides of the gripper member bracket 60 so as to provide a sensing beam across and in front of the finger members 64a,b. The left-front gripper member 42a is functionally similar to the right front gripper member 40a, except that it may not have a sensor system such as the photoelectric sensor system described above.

Whilst the front gripper members 40a, 42a are described as having two protrusions 62a,b and two movable finger members 64a,b, in other embodiments, there may be only one protrusion and one matching finger member or more than two protrusions and matching finger members. The finger members can be considered as comprising gripping means.

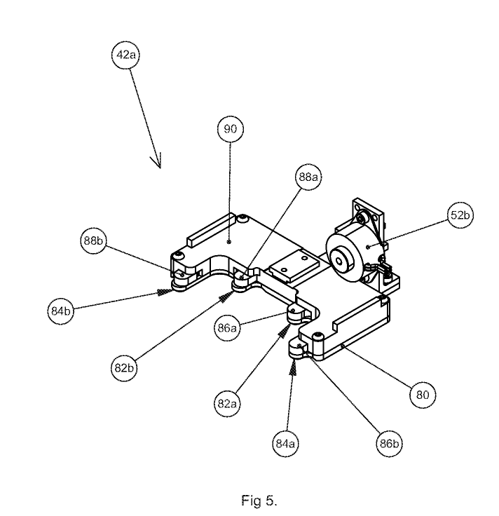

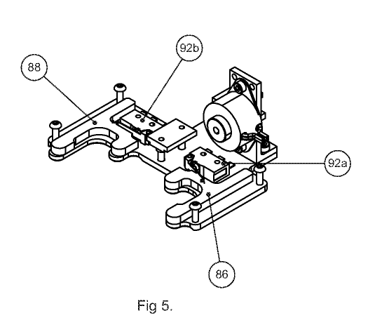

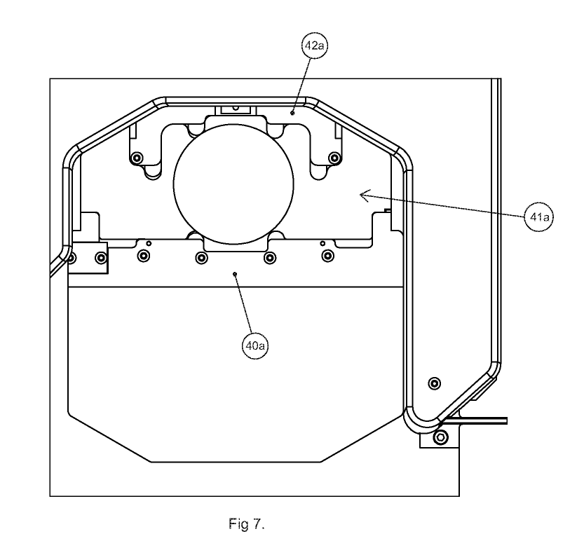

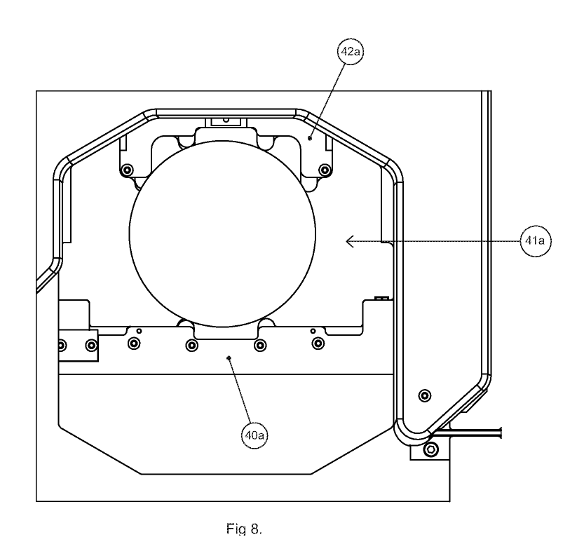

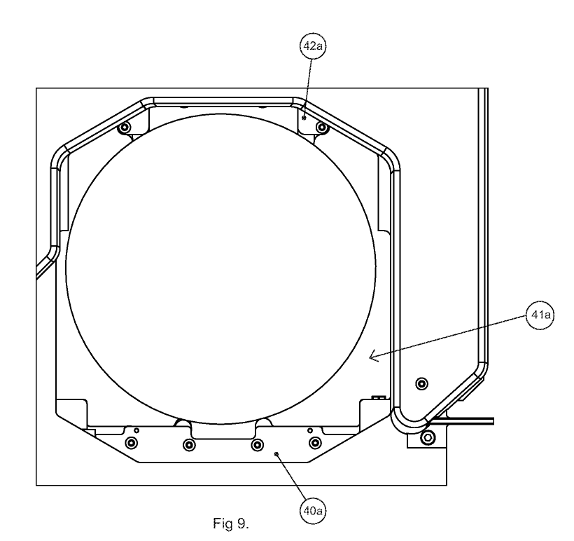

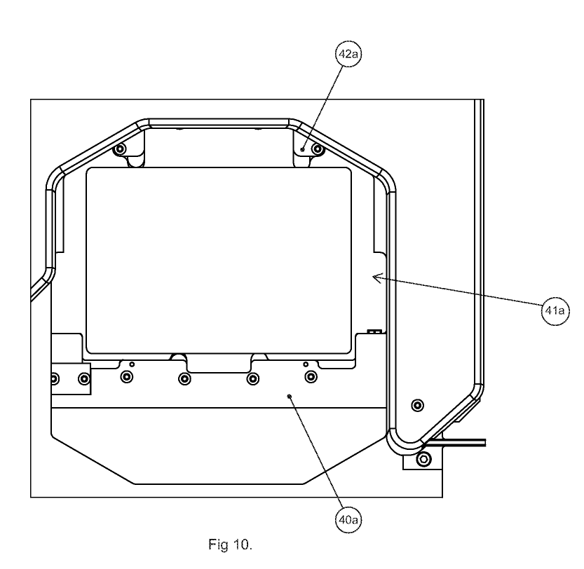

FIG. 5 shows the right side rear gripper member 40b which is generally C-shaped in plan. The left rear gripper member 42b is functionally similar to the right rear gripper member 40b. The right rear gripper member 40b comprises a plate support bracket 80 having four finger-like protrusions 82a,b, 84a,b: two on each side of the gripper member 40b. The protrusions 82a,b, 84a,b are arranged such that at least one protrusion on each side will support the lid of a media plate or a media plate itself of any plate type and/or plate size within a range of sizes that can be handled by the plate pourer apparatus 10. Rectangular, square and large-diameter round plates (Petri dishes) or lids are supported by the outer pair 84a,b of protrusions as illustrated by FIGS. 9 and 10 and small square or round plates or lids are supported by the inner pair of protrusions 82a,b as illustrated by FIG. 7. For example, the 60 mm diameter round plate or lid (FIG. 7) is supported by the inner pair of protrusions 82a,b whereas the 150 mm diameter round plate or lid (FIG. 9) is supported by the outer pair of protrusions 84a,b. Round plates or round plate lids in the size range of 90 to 100 mm in diameter may be supported by both pairs of protrusions 82a,b, 84a,b simultaneously (FIG. 8), depending on the actual diameter of the plate or lid.

Consequently, the gripper members 40a,b, 42a,b of the carriage 20 are adapted to accommodate not only different types and/or sizes of media plates/lids, but also handle variations in size of plates or lids of a specific type and/or size thereby making it easier to source plates/lids of a specific type and/or size from the consumables market. Similarly to the front gripper members 40a, 42a, the rear gripper members 40b, 42b have movable finger members that are pushed back when the gripper member engages a plate's lid or a plate's sides. In FIG. 5, it can be seen that the right rear gripper member 40b has two moveable finger members 86, 88 each with two protrusions 86a,b, 88a,b of matching shape to the protrusions 82a,b, 84a,b. Unlike the front finger members 64a,b which are pivot mounted, the rear finger members 86, 88 slide in elongated slots in a gripper member cover 90. There is only one finger switch 92a,b per side of the right rear gripper member 40b. Each finger switch 92a,b is activated by pushing on either the inner or outer protrusion of its movable finger member 86, 88. FIG. 5 shows the finger member 88 on the left hand side activated (pressed) and the finger member 86 on the right hand side released.

Whilst the rear gripper members 40b, 42b are described as having four protrusions 82a,b, 86a,b and four matching finger member protrusions 86a,b, 88a,b, the four finger member protrusions being arranged two to each of two slidable finger members 86, 88, it will be appreciated that in other embodiments the protrusions and matching finger member protrusions may be differently arranged. For example, it is only necessary for gripping different types and sizes of media plates for the rear gripper members to have at least one central protrusion and two outer protrusions arranged to either side of the at least one central protrusion with matching finger member protrusions, although it is preferred to have two central protrusions as depicted in FIG. 5. It is also necessary that the gripping ends of the two outer protrusions extend further into the gripping space 41a,b than the gripping end of the central protrusion. Furthermore, the matching finger member protrusions may be arranged on a single sliding finger member or provided on three respective slidable finger members with respective finger switches. Other arrangements are also possible.

Referring again to FIGS. 2 and 3 and FIG. 11, the liquid media dispensing system preferably comprises a nozzle arm 100 pivot mounted close to a centre of the carriage main plate member 50. The nozzle arm 100 extends below the carriage main plate member 50. The nozzle arm 100 is driven by a stepper motor 102 mounted underneath the carriage 20 through a belt-and-pulley arrangement 104 provided above the main plate 50. The nozzle arm 100 rotates directly underneath the gripper members 40a,b, 42a,b.

The location of the nozzle arm pivot point and the distance between the pivot point and the dispensing tip 101 of the nozzle arm 100 are such that the nozzle tip 101 is positioned close to the centre of each type and/or size of media plates by rotating the arm to a specific angular orientation relative to the carriage 20. The nozzle arm 100 draws media fluid or liquid such as an agar solution via an intake tube from a media flask in the apparatus 10.

A reflective optical encoder mounted on a PCB 106 above the nozzle arm pulley system 104 is used to find the arm's centre position and to measure its angular orientation when the stepper 102 motor is activated.

In another embodiment, a separate movable nozzle arm is provided for each side of the carriage 20, each nozzle arm being movable to a position such that a dispensing tip thereof is positioned over the basin of a media plate of any type and/or size arranged adjacent to or held in respective gripper members 40a,b, 42a,b of the carriage 20.

It will be understood that, whilst the carriage is described as having left and right side pairs of gripper members, the carriage 20 may have a single set or pair of gripper members, the apparatus 10 being arranged to process only a single stack of media plates. In another embodiment, the carriage may be adapted to have two, three or more sets of gripper members, the apparatus 10 being arranged to process one, two, three or more stacks of media plates simultaneously.

Referring to FIGS. 2 and 6, it can be seen that the carriage is supported on the three lead screws 22 which are arranged to the front-right, front-left and rear-centre of the frame of the main cabinet 12. The rotation of the three lead screws 22 by the elevator mechanism 16 is synchronized. Respective pulleys 110 of the three lead screws 22 are connected by a single timing belt 112 to the elevator stepper motor 114 mounted inside the control cabinet 18.

The carriage mounting brackets 21 rest on nuts 116 of the lead screws 22, but can separate slightly from the nuts 116 when the carriage 20 is raised or lowered. Respective switches 118 are provided to detect when any of the support brackets 22 separates from its corresponding nut 116, which indicates that the carriage 20 has crashed into an object and is unable to move farther down.

The control cabinet 18 contains most of the control electronics including the process controller 15, as well as the stepper motors that power the elevator mechanism 16 and a media fluid pump unit 17 for the media flask. The cabinet 18 has a power switch 13, a touch-screen 19 for user control inputs, the pump unit 17 and a liquid sensor unit 122 mounted on its front wall.

Referring to FIGS. 1 and 6, the base plate 14 of the plate pourer apparatus 10 rests on three adjustable feet 120, which are each height adjustable to enable the apparatus to be levelled. The two level indicators 32 on the base plate 14 are provided one at each corner of the main cabinet 12 such that an axis of each indicator 32 is parallel to a line connecting its corresponding front foot 120 and the rear foot 120 of the apparatus 10. Therefore, adjusting the foot 120 at one corner, only affects the reading of the level indicator 32 on the same side of the apparatus 10.

The plate pourer apparatus 10 is preferably fully automated, but can be arranged to run custom plate filling routines. To fill a batch of plates with media the operator inserts the intake tube of the nozzle arm 100 into the media flask, loads the plates into the main cabinet 12 and activates the apparatus 10. Under normal circumstances, no further action is required on the operator's part as the apparatus 10 automatically completes the plate filling/pouring process.

The operator can control the apparatus 10 through the touch-screen and receives feedback through the touch-screen.

After power-up the operator is prompted to start the initialization procedure of the apparatus 10. The apparatus 10 is configured to go through a sequence of moves to find the reference, i.e. home, positions of the moving stages of the apparatus 10, namely: carriage 20 at the top of the cabinet 12; all gripper members open; nozzle arm 100 in a centre position.

The apparatus 10 is configured to then ask the operator to confirm that the media fluid is ready and that a batch of plates have been loaded into the apparatus 10. Once confirmed, the apparatus 10 starts a new plate pouring run.

With reference to FIG. 11, the carriage 20 is caused to move down to the height in the plate stack where the lid of the lowermost plate is expected to be found. Both front gripper members 40a, 42a close until the plate detect sensor 68, 70, i.e. the photoelectric thru-beam sensor on the right-front gripper member 40a, is activated showing that the lid of the lowermost plate is detected. The position of the front gripper members 40a, 42a is registered for plate type and/or size identification purposes.

The carriage 20 is then caused to move down slightly until the plate detect sensor 68, 70 is released, which indicates the position of the bottom edge of the lid. The carriage height is further re-adjusted relative to the lid bottom edge to position the gripper members at the height suitable for engaging the plate lid. Both front gripper members 40a, 42a close again until their finger switches 66a,b are activated. At this point, the position of the front gripper members 40a, 42a is again registered for plate identification purposes. If the finger switches 66a,b on both right and left front gripper members 40a, 42a are activated, the apparatus 10 will simultaneously process two stacks of plates. If only the right-gripper finger switches 66a,b are activated, the apparatus 10 will process one stack of plates.

Once the front gripper members 40a, 42a are positioned for lifting the lids of the lowermost plates in their respective plate stacks, the rear gripper members 40b, 42b close (one if only one stack of plates has been detected) until their finger switches 92a,b are activated. The position of both front and rear gripper members 40a,b, 42a,b is then slightly re-adjusted to move the lids away from the stack holder guide rods 30 and/or to reduce deformation of the lids caused by the gripper action.

The apparatus 10 is now ready to pour a given quantity of media fluid into the basin of the lowermost plate(s). At this point, both registered positions of the front gripper members 40a, 42a are used to identify the type and/or size of plates being processed. A set of type-specific operating parameters such as media volume, pump speed, nozzle arm delivery location, etc. is selected based on the detected type and/or size of plates.

With front and rear gripper members 40a,b, 42a,b closed, the carriage 20 moves up, thus lifting the lid(s) of the lowermost plate(s) together with all empty plates of the stacks resting on them. This creates a space for the nozzle arm 100 to access the interiors of the plates between the plate and lid.

The nozzle arm 100 rotates to position the nozzle tip 101 close to the centre of one of the plates. The carriage 20 may be moved down to bring the nozzle tip 101 closer to the bottom of the plate before the pump causes the tip 101 of the nozzle arm 100 to dispense media fluid into the plate. Once that plate is filled, the carriage 20 moves up and the nozzle arm 100 pivots to position the tip 101 over the second plate where the dispensing sequence is repeated (if both stacks are present), or to the centre position of the carriage 20 (if only one stack is present).

The carriage 20 then moves down to deposit the lids back onto the plates and both front and rear gripper members 40a,b, 42a,b open to release the lid(s). The carriage advances to the next plate(s) up the stacks, where the sequence of finding the bottom edge of the lid(s), closing the front and rear gripper members, lifting the lid(s), dispensing the media and depositing the lid(s) back onto plates is repeated. The apparatus 10 continues to process the plate(s) until the top of the stack(s) is reached. At this point, the gripper members are open fully and the carriage 20 moves up to the parking position at the top of the main cabinet 12, although an alternative parking position may be below the stack base plate 26.

If the left stack 44b of plates is lower than the right stack 44a, the apparatus 10 will recognize this condition through feedback from the finger switches and will continue processing the remaining plates in the right stack 44a only.

Once all plates have been processed, the operator is alerted by an audio and/or visual indicator. The operator may then decide to load another batch of empty plates and initiate another pouring run, or to purge the media tube.

At the beginning of each pouring run and until the point when the plates have been identified and the apparatus 10 is cleared to go, the operator can activate or deactivate a UV lamp cycle-before-pouring, and automatic filling of the media tube (after the UV lamp cycle and before the pouring cycle). At any point during the pouring run the operator can modify any of the programmable parameters (e.g. dispensed volume, UV light timer). The new parameters become effective immediately once the changed settings are confirmed.

Whilst the above description of the operation of the apparatus is given with respect to self-supporting stacks of plates which have lids, it will be understood that the apparatus can be used to process self-supporting stacks of plates where the plates do not have lids, but where each upper plate rests on a rim of a lower plate. In this case, the above described operation is modified such that the carriage is initially moved to near the height of the lowermost plate by way of detecting the bottom of the next plate up which is resting on to rim of the lowermost plate. The gripping members are then controlled to engage the bottom of the next plate up from the lowermost plate in order to lift it and other plates in the stack to provide access to the interior of the lowermost plate for filling.

The apparatus 10 according to the invention provides a number of benefits over know plate pourer apparatuses in that it can handle rectangular and square plates in addition to round Petri dishes; it can handle a wide range of dimensional variations within each nominal size of plate; and it does not require any adjustments for, or even defining of the type of plate being processed. The apparatus 10 automatically detects the type of plates and performs pouring according to a specific pre-programmed set of parameters for the detected type and/or size of plate. It also maintains sterility throughout the pouring process with all plates being contained inside an enclosed cabinet with a programmable germicidal UV light procedure. Furthermore, spills are easy to clean, because the entire cabinet is easily accessible and there are no moving parts below the media dispensing nozzle arm. The apparatus 10 can produce excellent quality agar surface with a minimum meniscus as the already filled plates are not disturbed or moved while remaining plates in a stack are being filled.

In general, the invention discloses a method of filling media plates as follows: at least one stack of plates is loaded into the stack holders 24a,b of the apparatus 10. The vertically moving carriage 20 which contains plate gripper members 40a,b, 42a,b with sensors moves to the approximate location of the lowermost plate within the stack. One or more sensor(s) are used to locate the bottom edge of the lowermost lid within the stack by moving the carriage 20 up and down and by moving the sensor-equipped gripper members toward and away from the plate. Based on the position of the moving stages during the locating process, the controller 15 determines the type and/or size of plate being used and selects the set of pre-programmed parameters specific for the type and/or size of plate. The carriage 20 moves to a suitable elevation and the gripper members close to engage the lowermost lid(s). The carriage 20 moves up thus elevating the lowermost lid(s) together with all empty plates within the stack(s) that rest on the lowermost lid(s). The dispensing nozzle arm 100 enters the space between the lowermost plate and lid and a quantity of media (agar) is dispensed into the plate from the nozzle arm tip 101. If there is more than one stack of plates present, the nozzle arm 100 then moves over the next available plate and the media dispensing process is repeated. The carriage 20 moves down to deposit the lid(s) onto the plate(s) and the grippers open to disengage from the lids. The carriage 20 moves up to the approximate location of the next plate within the stack and the process of locating the edge of the lid(s), lifting the lid(s), dispensing media, lowering the lid(s) and disengaging the lid(s) is repeated until the top of the stack(s) is reached.

The entire working head of the apparatus 10 comprising the carriage 20 moves up and down a stationary stack (or stacks) of plates. The apparatus gets access to a plate by lifting its lid together with all plates resting on the lid. The method works without re-organizing plates in the stack(s) and without disturbing (moving) the already processed plates.

The carriage has a set of at least two gripper members 40a,b, 42a,b, each with one or more finger members to engage and support a lid (or plate) in a stack. The finger members are arranged such that at least three finger members engage and support each type and/or size of plate/lid. The support points are spaced around the circumference of the plate/lid to assure stability of the stack above. Each finger may have a sensor that indicates whether the finger is engaged with the plate/lid. At least one gripper member has a sensor 68, 70 that detects the presence of a lid/dish as the gripper approaches the stack. This sensor may be of a non-contact type such as a through-beam optical type. Each gripper member is preferably equipped with an overload-detect sensor.

The liquid sensor unit 122 may comprise a photoelectric thru-beam sensor which may be configured to detect three conditions: no media tube present; media tube empty; or media tube filled with liquid.

When an “empty tube” condition is detected during operation, the apparatus may be configured to a refill procedure comprising: run the pump unit 17 back by an intake section volume; ask operator to check/refill the media container; run the pump unit 17 forward by an intake section volume; check if the media tube full; if yes, run the pump unit 17 forward to refill the entire tube; if not, repeat the refill procedure.

The plate stack support units 24a,b with the guide rods 30 are removable from the main cabinet 14 without the need to use tools.

The carriage 20 is supported by three lead screws 22 synchronized by a common drive mechanism (timing belt) 112. The three lead screws provide both vertical and horizontal support to the carriage.

Sensors 118 at the support points of the carriage 20 detect no-load condition on each individual screw 22 for crash sensing.

While the invention has been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art.

1. A method of filling one or more of a plurality of media plates each with a given quantity of media fluid, said media plates being arranged in a self-supporting stack, the method comprising the steps of:
moving a carriage along a height of the stack of media plates to a position of a media plate to be filled;
causing gripping means of said carriage to grip a lid of said media plate or, if said media plate has no lid, to grip a media plate directly above said media plate to be filled;
causing said carriage to rise to lift said lid, if present, and lift any media plates arranged above said media plate to be filled to provide access to an interior of said media plate;
filing the interior of said media plate with a given quantity of media fluid;
causing said carriage to descend to replace said lid, if present, and return any lifted media plates to their former positions arranged above said media plate to be filled.

2. The method of claim 1, wherein it is not necessary to remove a media plate from the stack in order to gain access to its interior.

3. The method of claim 1, wherein it includes performing the steps of the method of claim 1 for each of a plurality of media plates in the stack.

4. The method of claim 1, wherein it includes performing the steps of the method of claim 1 for each of a plurality of media plates in the stack starting with the lowermost plate and moving to each plate in turn up the height of the stack.

5. The method of claim 1, wherein the method further comprises parking the carriage at a position above or below the stack of plates when a program of filling plates is completed.

6. The method of claim 5, wherein the carriage is provided with sufficient gripping means so as to perform the steps of the method of claim 1 simultaneously on two or more stacks of media plates.

7. The method of claim 6, wherein the filling step is modified so to fill corresponding plates in different stacks one after the other from a movable media fluid dispensing means.

8. The method of claim 1, wherein said carriage is provided with means for determining a size and/or a type of a media plate in the stack and the method further comprises determining an operating parameter for filling said media plate based on a determined size and/or type of said plate.

9. The method of claim 8, wherein said operating parameter comprises one or more of: volume of media to be filled in the plate; pumping speed for media filling; and location of delivery of media to the interior of the plate.

10. An apparatus for filling one or more of a plurality of media plates each with a given quantity of media fluid, said apparatus comprising:
a media plate stack support on which a plurality of media plates can be arranged in a self-supporting stack;
a carriage movable along a height of the stack of media plates to a position of a media plate to be filled, the carriage carrying gripping means for gripping a lid of said media plate or, if said media plate has no lid, to grip a media plate directly above said media plate to be filled, said carriage being arranged to rise to lift said lid, if present, and lift any media plates arranged above said media plate to be filled to provide access to an interior of said media plate; and
a media fluid dispensing means for filing the interior said media plate with a given quantity of media fluid;
wherein, once said dispensing means has dispensed a given quantity of media fluid to the interior of the media plate, said carriage is arranged to descend to replace said lid, if present, and return any lifted media plates to their former positions arranged above said media plate to be filled.

11. The apparatus of claim 10, wherein said carriage has means for determining a size and/or a type of a media plate to be filled.

12. The apparatus of claim 11, wherein a process controller of the apparatus is configured to determine an operating parameter for filling said media plate based on a determined size and/or type of said plate.

13. The apparatus of claim 12, wherein said operating parameter determined by said process controller comprises one or more of: volume of media to be filled in the plate; pumping speed for media filling; and location of delivery of media to the interior of the plate.

14. The apparatus of claim 10, wherein a process controller of said apparatus is configured to control said apparatus to perform the steps of the method of claim 1 for each of a plurality of media plates in the stack starting with the lowermost plate and moving to each plate in turn up the height of the stack.

15. The apparatus of claim 14, wherein the carriage has two or more gripping means for gripping lids of media plates, if present, or gripping next upper media plates in two or more respective stacks of media plates and the process controller is configured to perform the steps of the method of claim 1 simultaneously on said two or more stacks of media plates.

16. (canceled)

17. (canceled)

18. An apparatus for filling a media plate with a given quantity of media fluid having a process controller for controlling operations of the apparatus, the apparatus comprising:
means for automatically determining a size and/or a type of a media plate to be filed;
means for determining an operating parameter for filling said media plate based on the determined size and/or type of said plate; and
dispenser means for filling said media plate with a given quantity of media fluid in accordance with said determined operating parameter.

19. The apparatus of claim 18, wherein the means for automatically determining a size and/or a type of a media plate and the means for determining an operating parameter comprise:
gripping means of said apparatus configured to engage a lid of a media plate to be filled or to engage the media plate itself; and
a processor controller configured to receive an input signal from the gripping means and to determine from said input signal a size and/or a type of a media plate to be filled.

20. (canceled)

21. (canceled)

22. (canceled)
